(19) United States
(12) Patent Application Publication
(10) Pub. No.: US 2011/0236878 A1
(43) Pub. Date: Sep. 29, 2011

(54) Rapid Characterization of Proteins in Complex Biological Fluids

(76) Inventor: Ivan Correia, Winchester, MA (US)

(21) Appl. No.: 13/013,544

(22) Filed: Jan. 25, 2011

Related U.S. Application Data
(60) Provisional application No. 61/298,028, filed on Jan. 25, 2010.

(51) Int. Cl. C12Q 1/00; G01N 33/68; G01N 33/74; G01N 27/447

(52) U.S. Cl. 435/4; 436/86; 204/450; 204/451

(57) ABSTRACT

Disclosed herein are compositions and methods for the rapid screening of candidate protein therapeutics. In particular, the instant invention provides compositions and methods for assaying the behavior of candidate protein therapeutics in complex biological fluids and for identifying those candidate protein therapeutics exhibiting desirable pharmacokinetic properties in such fluids.

The instant application claims the benefit of the filing date of U.S. Provisional Application No. 61/298,028, filed Jan. 25, 2010, which is hereby expressly incorporated by reference in its entirety.

### FIELD OF THE INVENTION

The instant invention is directed to compositions and methods for the rapid screening of candidate protein therapeutics. In particular, the instant invention provides compositions and methods for assaying the behavior of candidate protein therapeutics in complex biological fluids and for identifying those candidate protein therapeutics exhibiting desirable pharmacokinetic properties in such fluids.

### BACKGROUND OF THE INVENTION

In developing biologics for therapeutic applications, it is important, at an early stage, to be able to assess the stability and activity of a candidate compound. When many candidates are to be tested, evaluation in vivo is impracticable. Accordingly, in vitro analytical techniques have been developed to provide preliminary stability and activity data. Such techniques, however, are generally conducted using standard buffers and well-defined conditions which approximate, but may be critically different from, the context of complex biological fluids in which the candidate compound must be stable and active to be therapeutically useful. Over the last few years it has become apparent that protein therapeutics, such as antibodies and other recombinantly-expressed polypeptides, exhibit altered physiochemical characteristics, including decreased potency, when recovered after circulation in blood. Thus, current initiatives in drug development that call for the rapid evaluation and selection of candidate protein therapeutics employing standard buffers and conditions have the potential to provide insufficient data to accurately assess clinical effectiveness and stability. In particular, conventional assays cannot discriminate between candidate protein therapeutics that, in complex biological fluids, are stable and show desirable pharmacokinetic (“PK”) properties from those that are unstable and perform poorly.

In one example of such conventional analytical techniques, Chen et al., Glycobiology, 19(3):240-249 (2009), describe the electrophoretic analysis of monoclonal antibodies present in human serum. The analytical technique employed by Chen et al. requires that, prior to electrophoretic separation of the antibodies of interest to identify certain physiochemical characteristics, those antibodies are first removed from the serum sample via affinity isolation and then resuspended in a standard phosphate buffered saline (“PBS”) solution. Thus, this technique is incapable of isolating the specific contributions on the physiochemical characteristics of the exposure to human serum versus the contributions of the affinity isolation and resuspension in PBS. Furthermore, the affinity isolation and resuspension steps add significant time and cost to the antibody characterization technique.

A high throughput technique capable of rapid, precise, and sensitive analysis of candidate protein therapeutics in complex biological fluids, such as blood, serum, plasma, lymph, urine, and saliva, would not only reduce development costs but would also facilitate the efficient screening of a large number candidate molecules. The present invention addresses these needs.

### SUMMARY OF THE INVENTION

In certain embodiments, the present invention is directed to methods for analyzing a physiochemical property of a candidate protein therapeutic, wherein the candidate protein therapeutic is first labeled, then exposed to a complex biological fluid, and thereafter a sample of the complex biological fluid comprising the labeled candidate protein therapeutic is obtained and separated based on a physical attribute of the candidate protein therapeutic, wherein the label is detected to determine a physiochemical property of the candidate protein therapeutic.

In certain embodiments, the label on the candidate protein therapeutic is a fluorescent label.

In certain embodiments the fluorescent label on the candidate protein therapeutic is Pico Protein® dye (Caliper Life Sciences, Inc., Hopkinton, Mass.).

In certain embodiments of the invention, the candidate protein therapeutic is an selected from the group consisting of an antibody, an antibody mimetic, such as an immunoadhesion molecule, an enzyme, a cytokine, a cytokine receptor, a lymphokine, a lymphokine receptor, and a hormone.

In certain embodiments, the physiochemical property to be assayed is selected from the group consisting of: (a) the fragmentation profile of the candidate protein therapeutic; (b) the propensity of the candidate protein therapeutic to aggregate; (c) the propensity of the candidate protein therapeutic to lose activity; and (d) another pharmacokinetic characteristic of the candidate protein therapeutic.

In certain embodiments the physiochemical property to be assayed is a pharmacokinetic characteristic of the candidate protein therapeutic that is selected from the group consisting of the rate of absorption, the rate of distribution, the rate of metabolism, the rate of excretion, the extent of absorption, the extent of distribution, the extent of metabolism and the extent of excretion of a candidate protein therapeutic.

In certain embodiments of the present invention the complex biological fluid to which the candidate protein therapeutic is exposed is selected from the group consisting of blood, plasma, serum, lymph, urine, and saliva.

In certain embodiments, the separation of the complex biological fluid comprising the candidate protein therapeutic is an electrophoretic separation.

In certain embodiments, the electrophoretic separation is capillary electrophoresis, such as the capillary electrophoresis performed using a LabChip® GXII instrument (Caliper Life Sciences, Inc., Hopkinton, Mass.).

### BRIEF DESCRIPTIONS OF THE DRAWINGS

FIG. 1 depicts a compilation of electropherograms illustrating the protein composition of mAb-1 samples at 5° C. and after heating at 25° C. and 40° C. for 6 and 3 months, respectively. “LC” refers to light chain; “HC” refers to heavy chain; “Fab” refers to the Fab fragment, which, as outlined in more detail in the detailed description of the invention, comprises the antigen binding region of the antibody; and “Fc” refers to the Fc fragment, which, as outlined in more detail in the detailed description of the invention, comprises the constant region of the antibody; and “Aggregate” refers to aggregations of antibodies.

FIG. 2 depicts a compilation of electropherograms illustrating the precision of the data (n=3) for mAb-1 obtained after 4 and 24 hours of incubation in whole blood.

FIGS. 3(A)-3(B) depict compilations of electropherograms obtained for the (A) DVD-1 and (B) mAb-1 molecules after incubation in whole blood for T=0 hrs, T=4 hrs and T=24 hrs.

### DETAILED DESCRIPTION OF THE INVENTION

The present invention is directed to compositions and methods for the rapid screening of candidate protein therapeutics. In particular, the instant invention provides compositions and methods for assaying the behavior of candidate protein therapeutics in complex biological fluids and for identifying those candidate protein therapeutics exhibiting desirable PK properties in such fluids.

For clarity and not by way of limitation, this detailed description is divided into the following sub-portions:
1. Definitions;
2. Properties to be Analyzed, and
3. Methods of Analysis.

1. Definitions

In order that the present invention may be more readily understood, certain terms are first defined.

The term “protein”, as used herein, is intended to refer to a composition comprising amino acid residues linked by peptide bonds. The term protein, as used herein, can be synonymous with the term “polypeptide” or can refer, in addition, to a complex of two or more polypeptides, such as an antibody comprising both heavy and light chain polypeptide molecules bound together by disulfide bridges.

The term “antibody”, as used herein, is intended to refer an immunoglobulin-containing molecule. An immunoglobulin is comprised of four polypeptide chains, two heavy (H) chains and two light (L) chains inter-connected by disulfide bonds. Each heavy chain is comprised of a heavy chain variable region (abbreviated herein as HCVR or V_{H}) and a heavy chain constant region (C_{H}). The heavy chain constant region is comprised of three domains, C_{H1}, C_{H2} and C_{H3}. Each light chain is comprised of a light chain variable region (abbreviated herein as LCVR or V_{L}) and a light chain constant region. The light chain constant region is comprised of one domain, C_{L}. The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. There are 5 classes of immunoglobulins, IgA, IgD, IgE, IgG, and IgM, with each class identified on the basis of the structure of their heavy chain constant region. The heavy chain constant regions of IgA, IgD and IgG each have three Ig domains and a hinge region to provide flexibility; whereas, the constant regions of IgE and IgM has four Ig domains. The IgA and IgG classes are further classified into two and four individual isotypes, respectively (IgA1 and IgA2; IgG1, IgG2, IgG3, and IgG4).

Certain antibody fragments are described herein as including the “antigen-binding portion” of an antibody (or “antibody portion”). These fragments include those portions of an antibody that retain the ability to specifically bind to an antigen. Examples of antigen binding fragments encompassed within the term “antigen-binding portion” of an antibody include (i) a Fab fragment, a monovalent fragment comprising the V_{L}, V_{H}, C_{L} and C_{H1} domains; (ii) a F(ab′)2 fragment, a bivalent fragment comprising two Fab fragments linked by a disulfide bridge at the hinge region; (iii) a Fd fragment comprising the V_{H} and C_{H1} domains; (iv) a Fv fragment comprising the V_{L} and V_{H} domains of a single arm of an antibody, (v) a dAb fragment (Ward et al., (1989) Nature 341:544-546, the entire teaching of which is incorporated herein by reference), which comprises a V_{H} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see, e.g., Bird et al. (1988) Science 242:423-426; and Huston et al. (1988) Proc. Natl. Acad. Sci. USA 85:5879-5883, the entire teachings of which are incorporated herein by reference). Such single chain antibodies are also intended to be encompassed within the term “antigen-binding portion” of an antibody. Other forms of single chain antibodies, such as diabodies are also encompassed. Diabodies are bivalent, bispecific antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen binding sites (see, e.g., Holliger, P., et al. (1993) Proc. Natl. Acad. Sci. USA 90:6444-6448; Poljak, R. J., et al. (1994) Structure 2:1121-1123, the entire teachings of which are incorporated herein by reference). Furthermore, dual variable domain antibodies (DVD-IgG) are dual-specific, tetravalent immunoglobulin G (IgG)-like molecules that can be engineered from any two monoclonal antibodies while preserving activities of the parental antibodies (see, e.g., Wu et al., (2007) Nature Biotechnology 25, 1290-1297). Still further, an antibody or antigen-binding portion thereof may be part of a larger immunoadhesion molecule, formed by covalent or non-covalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101, the entire teaching of which is incorporated herein by reference) and use of a cysteine residue, a marker peptide and a C-terminal polyhistidine tag to make bivalent and biotinylated scFv molecules (Kipriyanov, S. M., et al. (1994) Mol. Immunol. 31:1047-1058, the entire teaching of which is incorporated herein by reference). Antibody portions, such as Fab and F(ab′)2 fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques.

Certain antibody fragments discussed herein do not retain antigen binding capability, including, but not limited to, Fc antibody fragments. The term “Fc fragment”, as used herein, refers to an antibody fragment that is produced when an immunoglobulin molecule is digested with papain, and refers to a region of an immunoglobulin molecule that excludes the variable region (V_{L}) and the constant regions (C_{L}) of the light chain and the variable region (V_{H}) and the constant region 1 (C_{H1}) of the heavy chain.

The term “complex biological fluid”, as used herein, is intended to refer to any circulating or non-circulating biological fluid found in a subject, including, but not limited to blood, serum, plasma, lymph, urine, cerebrospinal fluid, and saliva. The complex biological fluid may be minimally processed prior to separation analysis; for example, cellular and/or gross particular elements may be removed by centrifugation (low speed or high speed) or filtration or precipitation, or the fluid may be diluted using standard buffers known in the art. Even after such processing the same is referred to as “complex biological fluid” herein.

The term “whole blood”, as used herein, is intended to refer to the fluid that circulates in the heart, arteries, capillaries, and veins of vertebrate animals carrying nourishment and oxygen to and bringing away waste products from all parts of the body, which is composed of many types of cells, proteins and salts. Issaq et al., Chem Rev 107(8):3601-3620 (2007). When the cells are removed from this fluid without clotting, the liquid portion left behind is “plasma”; however, if cells are removed in the absence of anti-coagulants, the liquid portion is then called “serum.” Serum differs from plasma in that fibrin as well as proteins that associate with fibrin are removed; serum is estimated to have about 3-4% less protein than plasma. Plasma typically contains about 22 proteins that make up 99% of the total protein content—they include albumin, total IgG, transferrin, fibrinogen, total IgA, alpha-2-macroglobulin, total IgM, alpha-1-anti-trypsin, C3 complement, haptoglobulin, alpha-1-acid glycoprotein, apolipoprotein-B, apolipoprotein-A1, lipoprotein (a), factor H, ceruloplasmin, C4 complement, complement factor B, pre-albumin, C9 complement, C1q complement and C8 complement. Anderson et al., Mol Cell Proteomics 3(4):311-326 (2004) and Anderson et al., Mol Cell Proteomics 1(11):845-867 (2002). The remaining 1% of plasma is composed of hundreds of micro-abundant proteins. Serum proteins, thus, present an extremely “crowded” environment where the excluded volume results in antibody therapeutics adopting a compact structure as well as showing enhanced association with antigen in serum. Demeule et al., Anal Biochem 388(2):279-287 (2009). Furthermore, high levels of albumin, cysteine, cystine, glutathione, homocysteine and other small thiols also present a “redox” environment that facilitates rearrangement of disulfide bonds in antibody therapeutics recovered from serum. Summa et al., Proteins 69(2):369-378 (2007); Soriani et al., Arch Biochem Biophys 312(1):180-188 (1994); Mills et al., J Lab Clin Med 135(5):396-401 (2000); Hildebrandt et al., Mech Ageing Dev 123(9):1269-1281 (2002); Fiskerstrand et al., Clin Chem 39(2):263-271 (1993); Di Giuseppe et al., J Lab Clin Med 142(1):21-28 (2003); and Andersson et al., Clin Chem 39(8):1590-1597 (1993).

The term “pharmacokinetic property” or “PK property”, as used herein, is intended to refer to a parameter that describes the disposition of a candidate protein therapeutic in a subject. Representative pharmacokinetic properties include, but are not limited to, the extent or rate of absorption, distribution, metabolism and excretion (“ADME characteristics”) of a candidate protein therapeutic in a subject.

The term “electrophoresis”, as used herein, is intended to refer to a technique in which an electromotive force (“EMF”) is used to push or pull molecules through a matrix, preferably through a polymeric matrix solution. The molecules are conventionally introduced into the matrix and, upon application of an electric current, the molecules will move through the matrix at different rates, towards the anode if negatively charged or towards the cathode if positively charged. Examples of electrophoretic matrices include polyacrylamide, as commonly used in SDS-PAGE separations, as well as polymer solutions with low viscosity for use in microfluidic electrophoretic separations, such as the capillary electrophoretic separations employed by the LabChip® GXIII instrument.

The term “subject”, as used herein, is intended to refer to any animal, including both human and non-human animals.

The term “about”, as used herein, is intended to refer to ranges of approximately 10-20% greater than or less than the referenced value. In certain circumstances, one of skill in the art will recognize that, due to the nature of the referenced value, the term “about” can mean more or less than a 10-20% deviation from that value.

2. Properties to be Analyzed

The analytical techniques of the instant invention can be employed to determine the behavior of a candidate protein therapeutic in a complex biological fluid as well as to identify one or more PK properties of the candidate protein therapeutic. For example, but not by way of limitation, the analytical techniques of the instant invention can be employed to determine certain molecular behaviors induced by exposure to complex biological fluids including, but not limited to, the fragmentation profile of a candidate protein therapeutic in such fluids, as well as the candidate therapeutic's propensity to form aggregates in such fluids. PK properties that can be evaluated using the analytical techniques of the instant invention include, but are not limited to the absorption, distribution, metabolism and excretion characteristics of the candidate protein therapeutic. In certain embodiments of the present invention, two or more of such fragmentation, aggregation, and ADME characteristics can be determined simultaneously or serially.

In certain embodiments, the analytical techniques of the instant invention are employed to determine the fragmentation profile of a candidate protein therapeutic in a complex biological fluid. In certain embodiments, the fragmentation profile can include information relating to intermolecular fragmentation, such as the separation of heavy and light chain polypeptides in an antibody. In certain embodiments, the fragmentation profile can include information relating to intramolecular fragmentation, such as the cleavage of an antibody by a protease to release Fc, Fv, Fab, and/or F(ab′)2 fragments or a subfragment that is less than a complete antibody, less than a complete single chain immunoglobulin, less that a complete Fc, Fv, Fab, or F(ab′)2 fragment, etc. In certain embodiments the fragmentation profile can include information relating to both inter- and intramolecular fragmentation. Furthermore, in certain embodiments, the analytical techniques of the instant invention allow for comparison of fragmentation profiles produced as a result of exposure of the candidate protein therapeutic to different samples of the same type of complex biological fluids, to different types of complex biological fluids, as well as comparisons of fragmentation profiles based on the length of exposure of the candidate protein therapeutic to such fluids.

In certain embodiments, the analytical techniques of the instant invention are employed to determine the propensity of a particular candidate protein therapeutic to aggregate in the presence of a complex biological fluid. Furthermore, in certain embodiments, the analytical techniques of the instant invention allow for comparison of a certain candidate protein therapeutic's propensity for aggregation as a result of exposure of the candidate protein therapeutic to specific complex biological fluids as well as comparisons based on the length of exposure of the candidate protein therapeutic to such fluids.

In certain embodiments, the analytical techniques of the instant invention are employed to determine the extent or rate of absorption, distribution, metabolism or excretion of the candidate protein therapeutic. Furthermore, in certain embodiments, the analytical techniques of the instant invention allow for comparison of a certain candidate protein therapeutic's extent or rate of absorption, distribution, metabolism or excretion as a result of exposure of the candidate protein therapeutic to specific complex biological fluids as well as comparisons based on the length of exposure of the candidate protein therapeutic to such fluids.

3. Methods of Analysis

Certain embodiments of the present invention are directed to methods for assaying a physiochemical characteristic of a candidate protein therapeutic that has been exposed to a complex biological fluid. As outlined in detail below, these methods embrace numerous techniques for assaying physiochemical characteristics, for example, but not limited to, electrophoretic separation, size exclusion chromatography, and affinity chromatography, and can involve either in vitro or in vivo exposure of the candidate protein therapeutic to the complex biological fluid.

In certain embodiments, the candidate protein therapeutic of interest is labeled prior to exposure to a complex biological fluid. In other embodiments, the candidate protein therapeutic is labeled after exposure to the complex biological fluid and optionally prior to or after electrophoresis. In particular embodiments, the label is a fluorescent dye, such as Pico Protein® dye. In other embodiments, the candidate protein therapeutic is radiologically or chemically or enzymatically or immunologically labeled. In certain embodiments the candidate protein therapeutic is not labeled and instead detected via an immunological interaction (e.g., the binding of an antibody specific to the candidate protein therapeutic), a receptor/ligand interaction, an enzymatic interaction, or any other protein:protein or chemical interaction sufficient to identify the candidate protein therapeutic of interest.

In certain embodiments the labeled candidate protein therapeutic is exposed to a complex biological fluid via the direct spiking of a sample of that fluid in vitro. In certain embodiments the complex biological fluid is blood, plasma, serum, lymph, urine, or saliva. In such embodiments, the sample can be incubated for specific durations under specific conditions, e.g., temperature, pressure, etc., as determined advantageous by one of skill in the art. Following such direct spiking, one or more aliquots of the sample comprising the candidate protein therapeutic in the complex biological fluid are drawn at one or more time points as determined advantageous by one of skill in the art. In certain embodiments the components of the aliquot(s) are then separated based on a particular physical characteristic of those components. In particular embodiments the separation is based on charge, while in other embodiments the separation is based on an alternative physical characteristic, such as size, pH, or affinity for a particular chromatographic substrate. In embodiments where the separation is based on charge, the separation can be effectuated by employing an electrophoresis step. In particular embodiments, capillary electrophoresis is employed to separate the components of the aliquot(s) by charge. In certain embodiments, analysis of the separation of the candidate protein of interest, including fragments and aggregates thereof, is achieved by detecting the label that was bound to the protein prior to exposure to the complex biological fluid.

In certain embodiments, the candidate protein therapeutic is administered to a subject (which may be a human or non-human subject) in order to expose the candidate protein therapeutic to a complex biological fluid in vivo. The candidate protein therapeutic can be administered to a subject in any of a variety of forms. These include, but are not limited to, liquid, semi-solid and solid dosage forms, such as liquid solutions (e.g., injectable and infusible solutions), dispersions or suspensions, tablets, pills, powders, liposomes and suppositories. The form depends on, e.g., the intended mode of administration and proposed therapeutic application. Typical compositions are in the form of injectable or infusible solutions, such as compositions similar to those used for passive immunization of subjects with antibodies. One mode of administration is parenteral (e.g., intravenous, subcutaneous, intraperitoneal, intramuscular). In one aspect, the candidate protein therapeutic is administered by intravenous infusion or injection. In another aspect, the candidate protein therapeutic is administered by intramuscular or subcutaneous injection.

Upon administration of the candidate protein therapeutic to the subject, samples may be drawn from the subject at one more time points for analysis. The sample to be drawn, e.g., blood, lymph, urine, or saliva, can be determined by one of skill in the art based on the particular candidate protein therapeutic of interest. In certain embodiments, the sample to be drawn is a blood sample. In particular embodiments the drawn blood will be further processed into a plasma sample. In alternative embodiments the drawn blood will be further processed into a serum sample. Furthermore, the timing of such sampling can be determined by one of skill in the art based on both the candidate protein therapeutic of interest as well as known characteristics of the complex biological fluids to be sampled. In certain embodiments, such sampling will occur on an hourly, every 12 hours, or on a daily basis.

In certain embodiments the sample drawn after administration of a candidate protein therapeutic will be directly subjected to analysis based on a physical characteristic of the components of that sample. In alternative embodiments the sample will be further processed prior to such analysis. For example, but not by way of limitation, the sample can be diluted using standard buffers known in the art. The sample can also, or alternatively, be subjected to centrifugation, e.g., a blood sample can be spun at about 2,000 rpm for 5 minutes to produce a serum supernatant. The sample can also, or alternatively, be exposed to neutralizing compounds, e.g., protease inhibitors, to inhibit protein degradation prior to such analysis.

In certain embodiments the components of the sample are analyzed by separating those components based on a particular physical characteristic. In particular embodiments the separation is based on charge, while in other embodiments the separation is based on an alternative physical characteristic, such as size, pH, or affinity for a particular chromatographic substrate. In embodiments where the separation is based on charge, the separation can be effectuated by employing an electrophoresis step. In particular embodiments, capillary electrophoresis is employed to separate the components of the sample by charge. In certain embodiments, analysis of the separation of the candidate protein of interest, including fragments and aggregates thereof, is achieved by detecting the label that was bound to the protein prior to exposure to the complex biological fluid.

In certain embodiments the above described analysis allows for the comparison of the behavior of two or more candidate protein therapeutics in one or more complex biological fluids as well as the comparison of one or more PK properties of the candidate protein therapeutics in such fluids. In particular embodiments, such comparisons allow for the ranking of various candidate protein therapeutics, including, but not limited to distinct clones of monoclonal antibodies or DVD-IgG molecules.

In certain embodiments, the present invention allows for the simultaneous analysis of two or more candidate protein therapeutics. In particular embodiments of the instant invention, the candidate protein therapeutics have distinct physical characteristics, for example, but not limited to, charge or size, which allow for simultaneous analysis of multiple candidate therapeutics. In alternative embodiments, the candidate protein therapeutics have distinct labels that allow for simultaneous analysis of multiple candidate therapeutics. For example, but not by way of limitation, a first candidate protein therapeutic can be labeled with a first fluorescent label that emits at a first wavelength while a second candidate protein therapeutic can be labeled with a second fluorescent label that emits at a second wavelength.

In certain embodiments, the present invention allows for analysis of the degradation pathway and metabolites of particular candidate protein therapeutics. In particular embodiments, specific metabolites of the particular candidate protein therapeutic are labeled and exposed to a complex biological fluid, either in vitro or in vivo. Analysis of samples of the complex biological fluid comprising the specific metabolites of the particular candidate protein therapeutic can provide information relating to the behavior of the metabolites of the candidate protein therapeutics in one or more complex biological fluids as well as one or more PK properties of the metabolites of the candidate protein therapeutics in such fluids

In certain embodiments, a LabChip® GXII instrument or equivalent device is employed to perform the analysis. In performing its analysis, the LabChip® GXII instrument employs traditional gel electrophoresis principles in a chip format. However, the chip format dramatically reduces separation time and provides automated sizing and quantification information in a digital format. The chip contains an interconnected set of microchannels that join the separation channel, which includes the separation matrix, and buffer wells. One of the microchannels is connected to a short capillary that extends from the bottom of the chip at a 90-degree angle. This capillary sips sample from the wells of a microplate during the assay

Once the channels are filled, the chip functions as an integrated electrical circuit in order to accomplish electrophoretic separation of the sample. The circuit is driven by the various electrodes in the electrode cartridge that contact solutions in the chip's wells when the chip holder is closed. The polymer matrix filling the assay channels is designed to sieve proteins by size as they are driven through it by means of electrophoresis, similar to using polyacrylamide gels. The complex biological fluid sample comprising the candidate protein therapeutic is then moved electrophoretically into the assay channel. As the fragments move down the assay channel, they separate by size, finally passing the laser that excites the fluorescent dye bound to the candidate protein therapeutic. The software plots fluorescence intensity versus time and produces an electropherogram for each sample.

### EXAMPLES

1. Analysis of Proteins in Whole Blood

This example describes the use of the LabChip® GXII instrument to analyze pre-labeled mAbs and DVD-IgG molecules recovered directly from whole blood. The LabChip® GXII has a sizing range from about 14 to about 200 kDa with about a ±10% sizing resolution. The assay has about a 4 log linear range from about 50 pg/μL to about 100 ng/μL. As detailed below, labeling of the molecules of interest was carried out with the Pico Protein® dye provided by the manufacturer. The labeled antibody was spiked into whole blood and prior to analysis the blood was spun and the supernatant collected for analysis on the LabChip® GXII. Aliquots of the supernatants were electrokinetically loaded into the capillary and separated in a 14 mm long separation channel that contained a polymer solution with low viscosity. Analysis of each sample was performed in about 40 seconds; directly from a 96 or 384 well plate. This study demonstrates the great precision of the assay, as well as the resolution and excellent sensitivity of the method.

The dye solution, Pico Protein® dye, and protein ladder were prepared as described by the manufacturer. The labeling buffer used was 0.5M sodium bicarbonate (pH 8.0). Two different molecules were used in this study, mAb-1 (a monoclonal antibody) and DVD-1 (a dual variable domain antibody). The antibodies were diluted to 2 mg/mL in labeling buffer and 8 μg was then incubated with 40 μM of the working dye solution (final concentration of the antibody was 0.8 mg/mL). The labeling reaction was allowed to proceed for 1 hour and was then quenched with 1M ethanolamine in 0.1M Tris (pH 7.0).

Each of the molecules were then spiked into human blood (final concentration of antibody was about 0.1 mg/mL) and incubated for various times (up to 24 hrs) at 5° C. An aliquot of blood was then spun at 2000 rpm for 5 minutes and 5 μL of serum was collected and diluted to 0.01 mg/mL using sample buffer provided by the manufacturer. The sample was heated to 75° C. for 5 minutes prior to analysis on the LabChip® GXII.

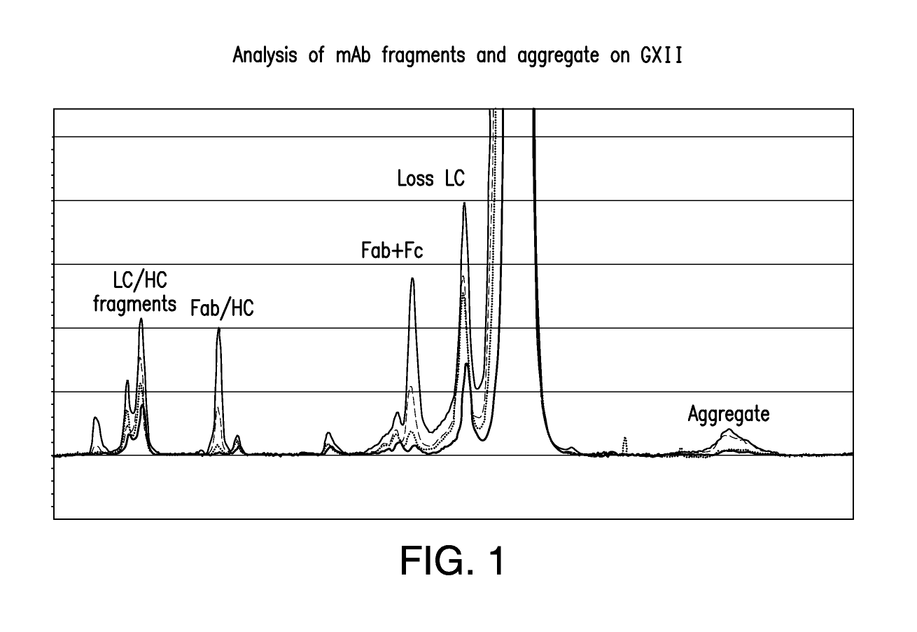

Shown in FIG. 1 are the analyses of mAb-1 at 5° C. and after heating at 25° C. and 40° C. for 6 and 3 months respectively. The electropherograms obtained are comparable to fragments analyzed by CE-SDS (capillary electrophoresis using denaturing sodium dodecyl sulfate) on other instruments. The LabChip® GXII showed all the known fragments obtained after heat stress as well as showed aggregation of the mAb-1.

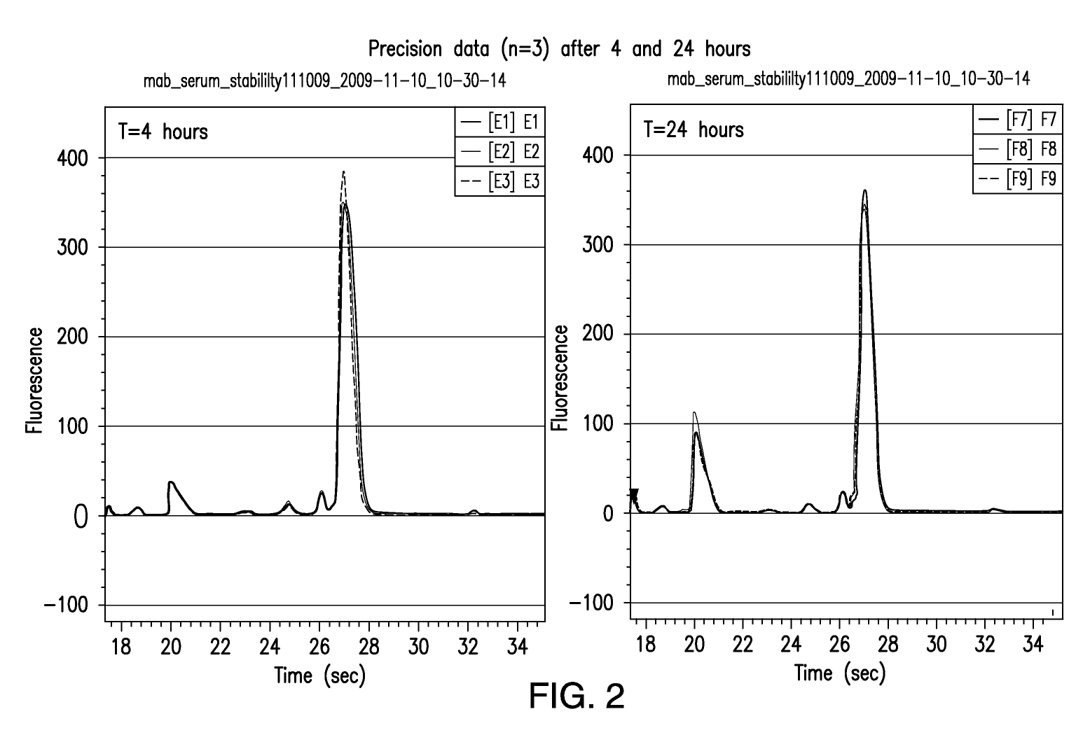

Shown in FIG. 2 is precision data (n=3) for mAb-1 obtained after 4 and 24 hours in whole blood. The electropherograms were obtained from 3 separate analyses and demonstrate excellent reproducibility of the assay.

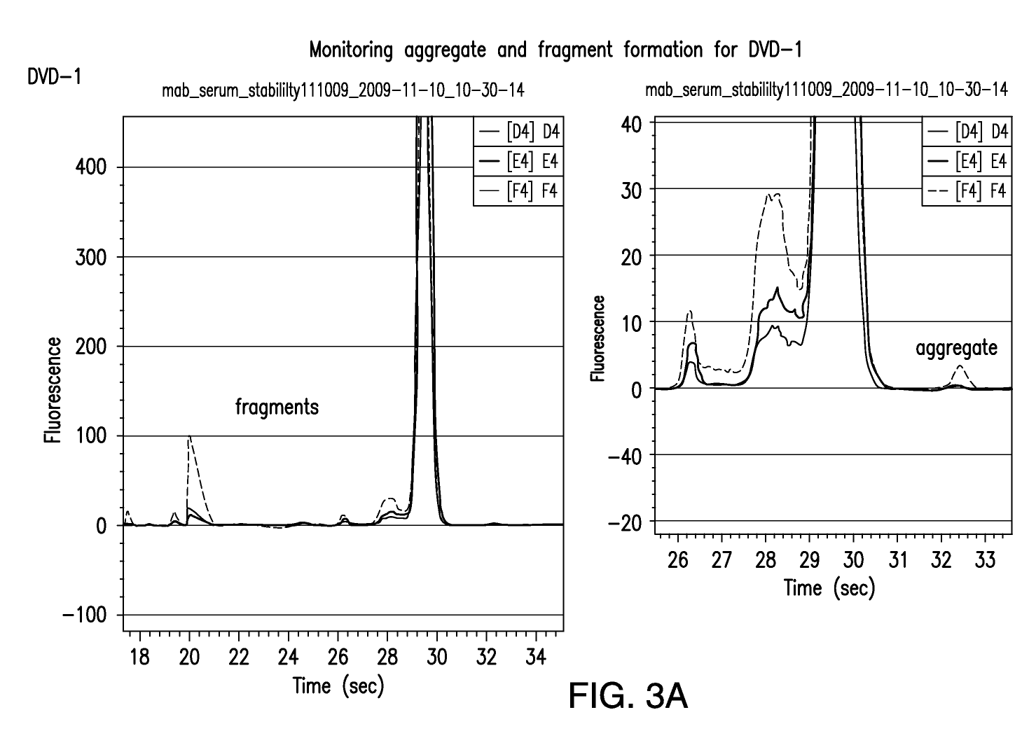

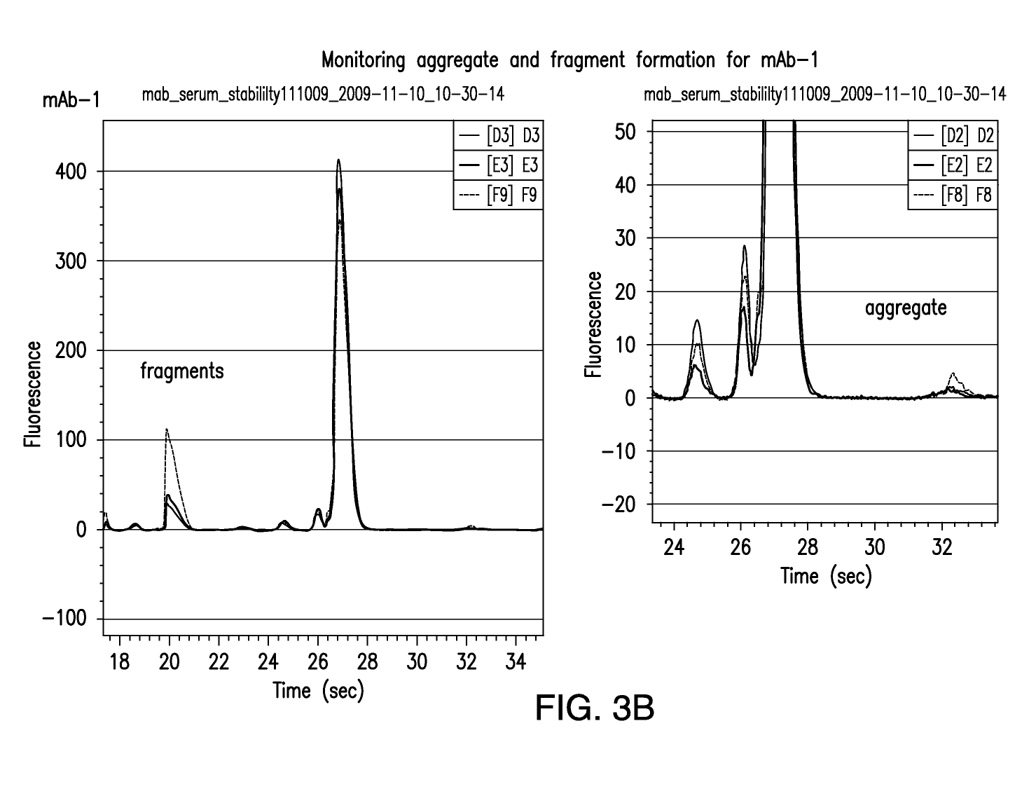

Shown in FIG. 3A and 3B are electropherograms obtained for the DVD-1 and mAb-1 molecules after incubation in whole blood for T=0 hrs, T=4 hrs and T=24 hrs. The DVD-1 molecule is larger and has a different migration time from the mAb-1. Both molecules show different levels of fragment and aggregate formation in whole blood.

2. Analysis of Proteins Administered to Animals

This example describes the use of the LabChip® GXII instrument to analyze pre-labeled mAbs and DVD-IgG molecules recovered directly from whole blood after administration to an animal subject. Labeling of the molecules of interest is carried out with the Pico Protein® dye provided by the manufacturer. The labeled antibody is administered to an animal subject and whole blood is obtained at various time points after such administration. Prior to analysis the blood is spun and the supernatant collected for analysis on the LabChip® GXII. Aliquots of the supernatants are electrokinetically loaded into the capillary and separated in a 14 mm long separation channel that contains a polymer solution with low viscosity. Analysis of each sample is performed in about 40 seconds; directly from a 96 or 384 well plate.

The dye solution, Pico Protein® dye, and protein ladder are prepared as described by the manufacturer. The labeling buffer used is 0.5M sodium bicarbonate (pH 8.0). Two different molecules are used in this study, mAb-1 (a monoclonal antibody) and DVD-1 (a dual variable domain antibody). The antibodies are diluted to 2 mg/mL in labeling buffer and 8 μg is then incubated with 40 μM of the working dye solution (final concentration of the antibody was 0.8 mg/mL). The labeling reaction is allowed to proceed for 1 hour and is then quenched with 1M ethanolamine in 0.1M Tris (pH 7.0).

Each of the molecules are then administered to a mouse (final concentration of antibody is about 0.1 mg/mL). Blood samples are drawn from the mouse daily for 14 days. An aliquot of each blood sample is then spun at 2000 rpm for 5 minutes and 5 μL of serum is collected and diluted to 0.01 mg/mL using sample buffer provided by the manufacturer. Each sample is heated to 75° C. for 5 minutes prior to analysis on the LabChip® GXII.

Various publications are cited herein, the contents of which are hereby incorporated by reference in their entireties.

What is claimed is:

1. A method for analyzing a physiochemical property of a candidate protein therapeutic, comprising:
(a) labeling said candidate protein therapeutic;
(b) exposing said labeled candidate protein therapeutic to a complex biological fluid;
(b) obtaining a sample of said complex biological fluid comprising said labeled candidate protein therapeutic;
(c) separating the components of said sample in a microfluidic device based on a physical attribute of the candidate protein therapeutic; and
(d) detecting said label to determine a physiochemical property of said candidate protein therapeutic in the separated sample.

2. The method of claim 1 wherein said label is a fluorescent label.

3. The method of claim 1 wherein said fluorescent label is Pico Protein dye.

4. The method of claim 1 wherein said candidate protein therapeutic is selected from the group consisting of an antibody, an antibody mimetic, an enzyme, a cytokine, a cytokine receptor, a lymphokine, a lymphokine receptor, and a hormone.

5. The method of claim 1 wherein said property is selected from the group consisting of: (a) the fragmentation profile of the candidate protein therapeutic; (b) the propensity of the candidate protein therapeutic to aggregate; (c) the propensity of the candidate protein therapeutic to lose activity, and (d) another pharmacokinetic characteristic of the candidate protein therapeutic.

6. The method of claim 5 wherein said property is the fragmentation profile of the candidate protein therapeutic.

7. The method of claim 5 wherein said property is the propensity of the candidate protein therapeutic to aggregate.

8. The method of claim 5 wherein said property is another pharmacokinetic characteristic of the candidate protein therapeutic.

9. The method of claim 8 wherein said pharmacokinetic characteristic is selected from the group consisting of the rate of absorption, the rate of distribution, the rate metabolism, the rate of excretion, the extent of absorption, the extent of distribution, the extent of metabolism and the extent of excretion of a candidate protein therapeutic.

10. The method of claim 1 wherein said complex biological fluid is selected from the group consisting of blood, plasma, serum, lymph, urine, cerebrospinal and saliva.

11. The method of claim 5 wherein said complex biological fluid is serum.

12. The method of claim 1 wherein said separation is electrophoretic separation.

13. The method of claim 12 wherein said electrophoretic separation is capillary electrophoresis.

14. The method of claim 13 wherein said capillary electrophoresis is performed using a LabChip® GXII instrument.
